(19) United States
(12) Patent Application Publication
(10) Pub. No.: US 2006/0264679 A1
(43) Pub. Date: Nov. 23, 2006

(54) Method for the production of tetrahydrofuran copolymers

(75) Inventors: Martin Haubner, Eppelheim (DE); Rolf Pinkos, Bad Durkheim (DE); Stephan Sclitter, Limburgerhof (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 10/551,142

(22) PCT Filed: Mar. 30, 2004
(86) PCT No.: PCT/EP04/03345

§ 371 Date: Sep. 29, 2005

(30) Foreign Application Priority Data
Apr. 1, 2003 (DE) 103 14 649.0

(51) Int. Cl. C07C 41/03

(52) U.S. Cl. 568/624

(57) ABSTRACT

The present invention relates to a process for the single-stage preparation of polyoxyalkylene glycols by copolymerization of THF and neopentyl glycol in the presence of a heteropolyacid, wherein the total amount of all impurities of the formula (I) 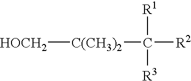
where R¹ and R² are each hydrogen when R³ is an oxyformyl or isopropionate radical, R¹ is hydrogen and R² is hydroxy when R³ is an isopropyl radical and R¹ is hydrogen when R² and R³ together form an —OCH₂—C(CH₃)—CH₂— radical, in the neopentyl glycol is less than 1000 ppm.

The present invention relates to a novel process for preparing polyoxyalkylene glycols (polyalkylene ether glycols) by copolymerization of tetrahydrofuran, hereinafter referred as “THF” for short, with neopentyl glycol in the presence of heteropolyacids, in which neopentyl glycol having a content of impurities of the formula I of less than 1000 ppm is used

Polyoxyalkylene glycols are important starting materials for the production of elastic fibers, elastic materials of construction and coatings. They can be prepared, inter alia, by polymerization of THF or by copolymerization of THF with neopentyl glycol, hereinafter referred to as “NPG” for short, in the presence of cationic catalysts. The use of heteropolyacids as catalysts is known from, for example, EP-A 126 471. This process makes it possible to obtain polyalkylene ether glycols in a single stage, while in other processes the esters of the polyoxyalkylene glycols are obtained first and have to be hydrolyzed to the polyoxyalkylene glycols before use in the field of polymers.

Technical-grade alpha, omega-diols such as neopentyl glycol contain small amounts of impurities in a concentration of up to 0.6%. Although this NPG is of very high purity, it has been recognized according to the present invention that the trace impurities present result in an unsatisfactory molecular weight and discoloration due to chain termination during the polymerization. Furthermore, it has at the same time been observed that the discoloration is associated with an altered reactivity of the copolymers in the preparation of polyesters and polyurethanes from the THF-NPG copolymers.

These are serious deficiencies, since color and reproducible processing are among the most important properties of a polymer which is to be used industrially.

It is an object of the present invention to provide a simple and inexpensive process for preparing THF copolymers with neopentyl glycol, which is suitable for preparing THF copolymers with neopentyl glycol having a low color number and makes do without an additional measure for decreasing the color number subsequent to the copolymerization.

We have found that this object is achieved by a process for the single-stage preparation of polyoxyalkylene glycols by copolymerization of THF and neopentyl glycol in the presence of a heteropolyacid, wherein the total amount of all impurities of the formula (I) 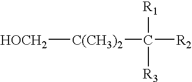
where R¹ and R² are each hydrogen when R³ is an oxyformyl or isopropionate radical, R¹ is hydrogen and R² is hydroxy when R³ is an isopropyl radical and R¹ is hydrogen when R² and R³ together form an —OCH₂—C(CH₃)—CH₂— radical,
in the neopentyl glycol is less than 1000 ppm, preferably less than 700 ppm, particularly preferably less than 500 ppm. ppm (parts per million) were determined by gas chromatography using a percent by area determination.

According to the present invention, it has been recognized that in the acid-catalyzed polymerization reaction, the compounds of the formula I act, inter alia, as polymerization termination reagents and stop chain growth and have an adverse effect on the color number of the copolymers. The process of the present invention makes it possible to prepare THF copolymers with neopentyl glycol having particular molecular weights of from 600 to 6000 dalton and a particular purity in a simple and reliable fashion.

Commercial technical-grade neopentyl glycol is treated by methods known per se to reduce the total content of impurities of the formula I to below 1000 ppm.

One way of purifying technical-grade neopentyl glycol for use in the process of the present invention is recrystallization of the neopentyl glycol from organic solvents.

Suitable organic solvents include C₁-C₁₀ alcohols such as methanol, ethanol, propanol or isopropanol, C₁-C₁₀ ethers such as tetrahydrofuran, diethyl ether, butylmethyl ether, and halogenated solvents such as chloroform or dichloromethane and mixtures thereof. Preference is given to using C₁-C₁₀ alcohols, particularly preferably methanol.

On a production scale, another possibility is the known layer or suspension crystallization method in which purification is achieved by crystallization form the melt and impurities remain in the melt.

Apart from recrystallization, the commercial neopentyl glycol can also be largely freed of the compounds of the formula (I) by catalytic hydrogenation.

The catalytic hydrogenation of the technical-grade neopentyl glycol can be carried out over catalysts of the Raney type, e.g. Raney nickel or Raney copper, or over supported copper, nickel or noble metal catalysts comprising elements of group VIII of the Periodic Table of the Elements, in particular platinum, or palladium catalysts.

Suitable support materials include all support materials known for hydrogenation catalysts used for the hydrogenation of carbonyl compounds, for example titanium dioxide, aluminum oxide, zirconium dioxide and zinc oxide. A preferred support material is a mixture of aluminum oxide, zinc oxide and, if desired, zinc-aluminum spinel. The preparation of the hydrogenation catalyst is not subject to any restrictions. It can be carried out by the methods customary for the preparation of such catalysts. The catalysts can be used as shaped bodies, for example as pellets, rings, angular pellets, other extrudate shapes, spheres or crushed material. The catalytic hydrogenation of neopentyl glycol is preferably carried out in a fixed-bed process, but a suspension process can in principle also be used.

A further method of purifying the neopentyl glycol used according to the present invention is solvent extraction of a saturated, aqueous, alcoholic or tetrahydrofuran-containing solution of neopentyl glycol by means of saturated or unsaturated aliphatic, cycloaliphatic or olefinic C₄-C₁₅-hydrocarbons or C₄-C₁₅-ethers. However, it is also possible to use hydrocarbons substance containing halogen atoms such as chlorine. Mixtures of the abovementioned classes of substance which have a proportion of at least 50% by weight of the hydrocarbon or ether can also be used.

On a production scale, liquid-liquid extraction can be carried out in a customary manner in one or more stages, generally in up to 5 stages. Suitable apparatuses and procedures are known to those skilled in the art and are described, for example, in “Ullmanns Encyclopedia of Industrial Chemistry, 6th Edition, Electronic Release”. Batchwise extractions can, for example, be carried out in a stirred vessel. Examples of continuous extraction are the use of sieve tray columns, stirred columns and extraction batteries, e.g. mixer-settlers. Membrane extractors, e.g. hollow fiber modules, can also be employed.

Among the purification methods suitable for removing the compounds of the formula I from the neopentyl glycol used according to the present invention down to less than 1000 ppm, preference is given to recrystallization.

According to the present invention, from 1 to 60% by weight of neopentyl glycol, based on the tetrahydrofuran used, preferably from 2 to 40% by weight, particularly preferably from 3 to 20% by weight, is used in the copolymerization.

Tetrahydrofuran is used in an amount of from 40 to 99% by weight, based on the total amount of THF and neopentyl glycol, preferably in an amount of from 60 to 98% by weight, particularly preferably from 80 to 97% by weight, in the copolymerization.

The copolymerization of THF with neopentyl glycol in the presence of heteropolyacids as catalysts is carried out in a manner known per se, as is described, for example, in EP-A 126 471.

The copolymerization of the present invention is preferably carried out in the presence of a hydrocarbon. A mixture of water and this hydrocarbon is distilled off from the copolymerization solution. For the purposes of the present patent application, the term “mixture” encompasses both customary nonazeotropic mixtures and hydrocarbon-water azeotropes. This mode of operation is described in the German patent application No. 102 399 47.6 dated Aug. 30, 2002 “Preparation of tetrahydrofuran copolymers” of BASF Aktiengesellschaft, which is hereby expressly incorporated by reference.

The hydrocarbons used should be suitable for forming an azeotrope with water. Hydrocarbons used are, for example, aliphatic or cycloaliphatic hydrocarbons having from 4 to 12 carbon atoms or aromatic hydrocarbons having from 6 to 10 carbon atoms or mixtures thereof. Specific mention may be made of, for example, pentane, hexane, heptane, octane, decane, cyclopentane, cyclohexane, benzene, toluene, xylene, or naphthalene, among which pentane, cyclopentane and octane are preferred and pentane is particularly preferred.

The hydrocarbons are added to the fresh feed to the copolymerization in an amount of from 1×10⁴⁻% by weight (corresponding to 1 ppm) to 30% by weight, based on the fresh feed comprising neopentyl glycol and THF, preferably from 1 ppm to 16% by weight, particularly preferably from 1 to 10% by weight. However, it is also possible to feed the hydrocarbon into the top of the distillation column for separating off the mixture of hydrocarbon and water. The molecular weight can be adjusted via the total amount of water which is removed from the copolymerization. In general, 1 mol of heteropolyacid binds from 10 to 40 molecules of water by coordination. The heteropolyacids used as catalysts should contain from about 1 to 10 molecules of water per molecule of heteropolyacid. In addition, water is liberated by the copolymerization with the neopentyl glycol used as comonomer. The higher the water content of the copolymerization solution, the lower the molecular weight of the copolymer obtained.

For the purposes of the present patent application, the term “mean molecular weight” or “mean molar mass” refers to the number average Mn of the molecular weight of the polymer molecules present in the polymer formed.

Heteropolyacids which are used according to the present invention are inorganic polyacids which, in contrast to isopolyacids, have at least two different central atoms. Heteropolyacids are formed from weak polybasic oxo acids of a metal, e.g. chromium, molybdenum, vanadium or tungsten, and a nonmetal, e.g. arsenic, iodine, phosphorus, selenium, silicon, boron or tellurium, as partial mixed anhydrides. Examples which may be mentioned are dodecatungstophosphoric acid H₃(PW₁₂O₄₀) and decamolybdophosporic acid H₃(PMo₁₂O₄₀). The heteropolyacids can also contain actinides or lanthanides as second central atom (cf. Z. Chemie 17 (1977), pages 353 to 357, and 19 (1979), 308). The heteropolyacids can generally be represented by the formula H₈₋ₙ(YⁿM₁₉O₄₀), where n=valence of the element Y (e.g. boron, silicon, zinc) (cf. Heteropoly- und Isopoly-oxomtalates, Berlin; Springer 1983). In the process of the present invention, phosphotungstic acid, phosphomolybdic acid, silicomolybdic acid and silicotungstic acid are particularly useful as catalysts.

The heteropolyacids used as catalysts can be used either in dried form (from 1 to 10 mol of water/mol of heteropolyacid) or undried form (from 10 to 40 mol of water/mol of heteropolyacid) in the copolymerization.

The water present in the copolymerization reactor, of which part is water of crystallization from the heteropolyacid and part is water formed during the reaction, is removed directly from the copolymerization reaction, i.e. from the copolymerization reactor without intermediate work-up steps such as phase separation, as a mixture of the hydrocarbon added with the fresh feed with water at from 40 to 120° C., particularly preferably from 50 to 70° C., and a pressure of from 150 mbar to 2 bar, preferably 230 mbar, with the aid of customary distillation apparatus.

The vapor formed is preferably condensed in a surface condenser; however, quench condensers and spray condensers are also possible. The condensate obtained is passed to solvent work-up for removal of the water from the system. It is particularly advantageous to recirculate part of the condensate to the reactor, i.e. to remove the heat of reaction by means of evaporative cooling. To achieve the highest possible water contents in the condensate to be taken off, a multistage countercurrent rectification column supplied with the return condensate as runback can be installed between reactor and condenser.

In a further embodiment, THF is distilled off simultaneously with the mixture of the hydrocarbon used in the copolymerization with water; the THF can, depending on the hydrocarbon, form a ternary azeotrope.

The hydrocarbon distilled off in a mixture with water or the mixtures of water and hydrocarbon with tetrahydrofuran can be dried by means of a suitable solid adsorbent, for example over molecular sieves, and be fed back into the copolymerization. Phase separation into an aqueous phase and the hydrocarbon is also conceivable. The aqueous phase contains up to 5% by weight of THF, preferably <1% by weight. Furthermore, the respective hydrocarbon is present in it in concentrations of <1% by weight. THF and the hydrocarbon can be recovered from the aqueous phase by distillation and be recirculated. However, the aqueous phase can also be discarded.

The copolymer solution remaining after the hydrocarbon/water mixture has been separated off is preferably passed to a phase separator. Addition of further amounts of hydrocarbon allows the heteropolyacid to be separated off from the product phase. This method which is known per se, for example from EP-A 181 621, leads to precipitation of the heteropolyacid from the organic phase. As hydrocarbon, preference is given to using the hydrocarbon which has been used in the copolymerization. The heteropolyacid is preferably reused for the next copolymerization.

The process of the present invention can be carried out either continuously or batchwise or as a semibatch process. A semibatch or semicontinuous mode of operation is one in which the heteropolyacid is initially charged together with 20-50% by weight of the other starting materials. The remainder of the starting materials is then metered in during the reaction time. In continuous and batch processes, the heteropolyacid is advantageously used in amounts of from 1 to 300 parts by weight, preferably from 5 to 150 parts by weight, based on 100 parts by weight of the monomers used (THF and alpha, omega-diols). It is also possible to add larger amounts of heteropolyacid to the reaction mixture.

The heteropolyacid can be introduced into the reaction in solid form, whereupon it is gradually solvated by being brought into contact with the further reactants to form the liquid catalyst phase. An alternative procedure is to slurry the solid heteropolyacid with the alpha, omega-diol and/or the THF to be used and to introduce the resulting catalyst solution into the reactor as a liquid catalyst phase. Either the catalyst phase or the monomeric starting material can be placed in the reactor first. However, it is also possible to introduce both components simultaneously into the reactor.

In a continuous mode of operation, water is metered into the reactor in an amount of from 0.1 to 5% by weight, preferably 0.1-3.5% by weight, particularly preferably 0.1-[lacuna] % by weight, based on the total amount of monomeric THF and comonomer usually via a level regulator. Fresh monomer is advantageously introduced in an amount corresponding to that in which product and unreacted monomer are discharged from the reaction apparatus. The residence time and therefore the polymerization time can also be controlled in this way, thus providing a further means of influencing and setting the mean molecular weight and the molecular weight distribution of the polymer formed.

The copolymerization can be monitored and controlled by means of an on-line conductivity measurement.

In a batch process, the copolymerization is preferably stopped in a conductivity range of 0.1-2.5 μS depending on the desired target molecular weight. To improve the stability of the organic product phase to oxidation, 10-500 ppm, particularly preferably 50-300 ppm, of a free radical scavenger can be added thereto. 250 ppm of 2,6-di-tert-butyl-4-methylkresol (BHT) are particularly suitable as free radical scavenger.

The control of the mean molecular weight via the electrical conductivity of the copolymer solution is disclosed in detail in the German patent application DE 10259036.2 dated Feb. 17, 2002, of the applicant, which is hereby expressly incorporated by reference.

In general, the copolymerization in a batch process is, depending on the amount of catalyst and the reaction temperature, carried out for a period of from 0.5 to 70 hours, preferably from 5 to 50 hours and particularly preferably from 10 to 40 hours. In a continuous process, residence times of from 1 to 50 hours, preferably from 10 to 40 hours, are usually set. At the beginning of a continuous reaction, the reaction system described takes some time until a steady-state equilibrium has been established and during this time it can be advantageous to keep the reactor outlet closed, i.e. not to discharge any product solution from the reaction apparatus.

The copolymerization is usually carried out at from 20 to 100° C., preferably from 30 to 80° C. It is advantageously carried out under atmospheric pressure, but a reaction under pressure, usually the autogenous pressure of the reaction system, can likewise prove to be useful and advantageous.

Whether the process is carried out batchwise, semicontinuously or continuously, the reactors should be equipped with effective mixing devices, for example stirrers. Suitable reactors include all liquid reactors which are known to those skilled in the art and have an internal or/and external free surface area for the necessary evaporation of the water-containing vapor and in which sufficiently high shear forces for suspending the catalyst phase in the homogeneous monomer/polymer phase are achieved in the liquid (stirred vessels, circulation reactors, jet loops, pulsed internals). A particularly advantageous type of construction is as a jet loop, since the necessary heating of the reactor can be integrated into the liquid circuit in a simple fashion. The water/hydrocarbon mixture is vaporized continuously or batchwise from the reaction mixture and the water content of the contents of the reactor is thus set to values which are advantageous for the reaction.

The process of the present invention is advantageously carried out under an inert gas atmosphere. Any inert gases such as nitrogen or argon can be used for this purpose. The reactants are likewise freed of any water and peroxides present therein before use.

In a continuous mode of operation, the reaction can be carried out in conventional reactors or reactor assemblies which are suitable for continuous processes, for example in tube reactors fitted with internals which ensure good mixing of the emulsion-like copolymerization mixture or else in cascades of stirred vessels.

For the present purposes, an emulsion-like copolymerization mixture is a mixture containing from 2 to 10 mol of water per mole of heteropolyacid.

The process of the present invention enables copolymers of THF and neopentyl glycol to be obtained economically and in good yield, selectively and with a narrow molecular weight distribution and also in pure form with a low color number. The copolymers have degrees of incorporation of the neopentyl glycol comonomer of from 5 to 50% by weight, based on the copolymer, and mean molecular weights Mn of from 600 to 6000. The polyoxyalkylene glycols which can be prepared according to the present invention are used, for example, for producing special polyurethanes which are suitable as high-elasticity composite materials. A polyurethane polymer derived from the copolymers which can be prepared according to the present invention has a high elongation to break, a low stress change during elongation, a small hysteresis loss on stretching and recovery and a high elasticity even at very low temperatures.

### EXAMPLES

Determination of the Color Number

The polymers which have been freed of the solvent are measured untreated in a liquid color measurement apparatus LICO 200 from the company Dr. Lange. Precision cells model no. 100-QS (path length: 50 mm, from Helma) are used.

Determination of the OH Number

The hydroxyl number is reported as the amount of potassium hydroxide in mg which is equivalent to the amount of acidic acid bound in the acetylation of 1 g of substance.

The hydroxyl number is determined by esterification of the hydroxyl groups present with an excess of acetic anhydride. After the reaction, the excess acetic anhydride is hydrolyzed with water and backtitrated as acetic acid with sodium hydroxide.

Determination of the Conductivity

Electrode: LTA 01 Glass/Platinum 2-electrode measurement cell, K about 0.1 cm⁻¹; Knick Conductometer (evaluation unit): Knick 702 from WTW (Wissenschaftlich technische Werkstätten).

The measurement apparatus firstly calculates the conductance of the solution from the measured current on the basis of Ohm's law and, using the cell constant, the conductivity value. Temperature compensation is carried out manually on the evaluation unit.

Determination of the Purity of the NPG by Gas Chromatography

Principle:

The sample for analysis is dissolved in methanol (solvent, e.g. Merck, Catalogue No. 106002) and analyzed by capillary gas chromatography. The chromatographic separation occurs on a dimethylpolysiloxane-coated fused silica capillary. A flame ionization detector (FID) is used for detection. Quantification is carried out by the percent by area method. The method serves to determine the main and secondary components in NPG in a range from 0.01 to >99% by area. A capillary gas chromatograph with automatic sample introduction unit, split injector and flame ionization detector (FID), e.g. HP 5890 with Autosampler HP 7673 A (Agilent), fused silica capillary coated with 100% dimethylpolysiloxane, e.g. RTX 1 from Restek (length: 30 m, internal diameter: 0.32 mm, film thickness: 1 μm), is used.

As integrator or computer with a suitable evaluation program, it is possible to use, for example, a VG-Multichrom (Labsystems);

Procedure

Sample Preparation

About 150 mg of polymer were dissolved in 1.5 ml of methanol. This solution was used directly for injection into the GC.

Chromatographic conditions

| | |
|---|---|
| Temperatures: | |
| Injector: | 300° C. |
| Column oven: | 80° C., 10 min isothermal |
| | 80° C. = 300° C., 5 K/min |
| | 300° C., 10 min isothermal |
| Detector (FID): | 320° C. |
| Carrier gas: | Nitrogen |
| Admission pressure to column: | 0.8 bar |
| Split: | 40 ml/min |
| Septum flush: | 3 ml/min |
| Combustion gases for FID: | Hydrogen and synthetic air, |
| | adjusted according to |
| | instructions of the manufacturer |
| Injection volume: | 1.0 μl |
| Calculation | $Fl - \% by area ( i ) = \frac{A ( i )}{\sum A} * 100$ |
| A(i) = | Peak area for the component i |
| | [μV · sec] |
| ΣF = | Sum of all peak areas of the |
| | components considered |
| | (subtract signals of the solvent) |
| | [pV · sec] |

### Examples 1 to 4 According to the Present Invention

1000 g of neopentyl glycol (commercial product of Mitsubishi Gas Chemical, containing more than 1000 ppm of compounds of the formula I, specifically 400 ppm of 2,2-dimethylpropane-1,3-diol monoformiate, 700 ppm of 2,2,4-trimethyl-1,3-pentandiol, 1900 ppm of neopentyl glycol isobutyrate, 300 ppm of β,β,5,5-tetramethyl-m-dioxane-2-ethanol) were admixed with 150 g of MeOH and the mixture was heated to 60° C. After complete dissolution, the source of heat was switched off and the solid crystallized over a period of 24 hours. The solid was filtered off, washed with cold MeOH and dried in a desiccator at 650° C. and a pressure of 5 mbar until the MeOH content was <10 ppm. The neopentyl glycol obtained contained the following amounts of compounds of the formula I: 40 ppm of 2,2-diemthylpropane-1,3-diol monoformiate, 80 ppm of 2,2,4-trimethyl-1,3-pentandiol, 270 ppm of neopentyl glycol isobutyrate, 0 ppm of β,β,5,5-tetramethyl-m-dioxane-2-ethanol.

In a 1 l double-walled reactor provided with a magnetic stirrer and connected distillation column (30 theoretical plates) with combined water separator, a mixture of 400 g of THF, 24 g of purified neopentyl glycol and 30 g of pentane was stirred until a homogenous solution had been formed. While stirring, 100 g of a hydrated dodecaphosphortungstic acid (commercial product from Merck, Darmstadt, water content not more than 17%) were added thereto. The reaction temperature was maintained in a range from 65 to 67° C.

The THF/pentane/water mixture which vaporized during the reaction was fractionated in the column. The pentane/water mixture was separated off at the top and condensed in the water separator. The bottoms from the column comprised mainly THF and were returned to the polymerization stage. The pentane/water mixture separated into two phases, of which the upper organic phase was recirculated to the top of the column. The lower aqueous phase was discarded.

After 22 hours, the reaction was stopped by addition of 10 g of water and 250 ppm of 2,6-di-tert-butyl-p-cresol (BHT) and 450 g of hexane. After phase separation, the lower aqueous phase was drained off.

The upper phase was passed at 20° C. over a fixed bed composed of a cation exchanger and an anion exchanger (volume: 1 l in each case) of the grade Lewatit® MP 600 R from Bayer, Leverkusen.

THF and heptane were then separated off on a rotary evaporator at 140° C. and a pressure of 20 mbar to give a copolymer having an OH number of 60 mg of KOH/g of copolymer. Further data are shown in table 1.

The aqueous heteropolyacid phase separated off in example 1 was used in example 2. Example 2 was carried out in a manner analogous to example 1. Further data are shown in table 1. After a reaction time of 21 hours, the reaction was stopped by addition of water and BHT.

The aqueous heteropolyacid phase separated off in example 2 was used in example 3. Example 3 was carried out in a manner analogous to example 1. Further data are shown in table 1. After a reaction time of 22 hours, the reaction was stopped as in example 1.

The aqueous heteropolyacid phase separated off in example 3 was used in example 4. Example 4 was carried out in a manner analogous to example 1. Further data are shown in table 1. After a reaction time of 22 hours, the reaction was stopped as in example 1.

The color number of the first experiment and the two subsequent experiments is <10 Apha.

Further data are listed in table 1.

TABLE 1

| | THF | NPG | HPA¹⁾ phase | Temp. | Reaction time | EVR²⁾ | OH | Color |
|---|---|---|---|---|---|---|---|---|
| Ex. | [g] | [g] | [g] | [° C.] | [h] | [%] | number | number |
| 1 | 400 | 24 | 100 | 65 | 21 | 34.5 | 60 | 10 |
| 2 | 400 | 24 | 148 | 65 | 21 | 34.8 | 62 | 9 |
| 3 | 400 | 24 | 153 | 66 | 22 | 35 | 60 | 10 |
| 4 | 400 | 24 | 150 | 66 | 22 | 35.2 | 61 | 10 |

¹⁾HPA = Heteropolyacid
²⁾EVR = Evaporation residue

### Comparative Examples 5 to 10

Example 5 was carried out in a manner analogous to example 1, but using unpurified NPG (Mitsubishi Gas Chemical; purity: 99.6% by area, more than 1000 ppm of impurities of the formula I, composition as reported in example 1).

The aqueous catalyst phase obtained was in each case reused in the subsequent example. A total of 5 repetitions were carried out. Further data are listed in table 2.

TABLE 2

| | THF | NPG | HPA¹⁾ phase | Temp. | Reaction time | EVR²⁾ | OH | Color |
|---|---|---|---|---|---|---|---|---|
| Ex. | [g] | [g] | [g] | [° C.] | [h] | [%] | number | number |
| 5 | 400 | 24 | 100 | 65 | 21 | 34.8 | 60 | 10 |
| 6 | 400 | 24 | 150 | 65 | 21 | 34.3 | 61 | 11 |
| 7 | 400 | 24 | 142 | 66 | 22 | 34.0 | 60 | 15 |
| 8 | 400 | 24 | 120 | 66 | 22 | 33.7 | 61 | 23 |
| 10 | 400 | 24 | 153 | 66 | 22 | 32.4 | 63 | 49 |
| 11 | 400 | 24 | 148 | 66 | 22 | 32.0 | 65 | 63 |

¹⁾HPA = Heteropolyacid
²⁾EVR = Evaporation residue

When unpurified neopentyl glycol is used, a significant rise in the color number is observed. Furthermore, there is a slight increase in the OH number owing to the forced chain termination caused by impurities of the formula I.

1. A process for the single-stage preparation of polyoxyalkylene glycols comprising copolymerization of THF and neopentyl glycol in the presence of a heteropolyacid, wherein the total amount of all impurities of the formula (I) 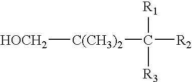
where R¹ and R² are each hydrogen when R³ is an oxyformyl or isopropionate radical, R¹ is hydrogen and R² is hydroxy when R³ is an isopropyl radical and R¹ is hydrogen when R² and R³ together form an —OCH₂—C(CH₃)—CH₂— radical,
in the neopentyl glycol is less than 1000 ppm.

2. The process for the single-stage preparation of polyoxyalkylene glycols as claimed in claim 1, wherein the content of impurities of the formula (I) in the neopentyl glycol is less than 700 ppm.

3. The process for the single-stage preparation of polyoxyalkylene glycols as claimed in claim 1, wherein the content of compounds of the formula (I) in the neopentyl glycol is achieved by recrystallization, solvent extraction or hydrogenation of technical-grade neopentyl glycol.

4. The process for the single-stage preparation of polyoxyalkylene glycols as claimed in claim 1, wherein from 3 to 20% by weight of neopentyl glycol, based on tetrahydrofuran, is used.

5. The process for the single-stage preparation of polyoxyalkylene glycols as claimed in claim 1, wherein the copolymerization is carried out in the presence of a hydrocarbon.

6. The process for the single-stage preparation of polyoxyalkylene glycols as claimed in claim 1, wherein the process is carried out continuously or batchwise.

7. The process for the single-stage preparation of polyoxyalkylene glycols as claimed in claim 1, wherein the copolymerization is carried out at from 20 to 100° C.

8. The process for the single-stage preparation of polyoxyalkylene glycols as claimed in claim 2, wherein the content of compounds of the formula (I) in the neopentyl glycol is achieved by recrystallization, solvent extraction or hydrogenation of technical-grade neopentyl glycol.

9. The process for the single-stage preparation of polyoxyalkylene glycols as claimed in claim 2, wherein from 3 to 20% by weight of neopentyl glycol, based on tetrahydrofuran, is used.

10. The process for the single-stage preparation of polyoxyalkylene glycols as claimed in claim 3, wherein from 3 to 20% by weight of neopentyl glycol, based on tetrahydrofuran, is used.

11. The process for the single-stage preparation of polyoxyalkylene glycols as claimed in claim 2, wherein the copolymerization is carried out in the presence of a hydrocarbon.

12. The process for the single-stage preparation of polyoxyalkylene glycols as claimed in claim 3, wherein the copolymerization is carried out in the presence of a hydrocarbon.

13. The process for the single-stage preparation of polyoxyalkylene glycols as claimed in claim 4, wherein the copolymerization is carried out in the presence of a hydrocarbon.

14. The process for the single-stage preparation of polyoxyalkylene glycols as claimed in claim 2, wherein the process is carried out continuously or batchwise.

15. The process for the single-stage preparation of polyoxyalkylene glycols as claimed in claim 3, wherein the process is carried out continuously or batchwise.

16. The process for the single-stage preparation of polyoxyalkylene glycols as claimed in claim 4, wherein the process is carried out continuously or batchwise.

17. The process for the single-stage preparation of polyoxyalkylene glycols as claimed in claim 5, wherein the process is carried out continuously or batchwise.

18. The process for the single-stage preparation of polyoxyalkylene glycols as claimed in claim 2, wherein the copolymerization is carried out at from 20 to 100° C.

19. The process for the single-stage preparation of polyoxyalkylene glycols as claimed in claim 3, wherein the copolymerization is carried out at from 20 to 100° C.

20. The process for the single-stage preparation of polyoxyalkylene glycols as claimed in claim 4, wherein the copolymerization is carried out at from 20 to 100° C.
